(19) United States
(12) Patent Application Publication
(10) Pub. No.: US 2026/0157769 A1
(43) Pub. Date: Jun. 11, 2026

(54) DEVICE AND KIT FOR HARVESTING TENDON TISSUE

(71) Applicant: Arthrex Inc., Naples, FL (US)

(72) Inventors: David Hegg, Naples, FL (US); Samuel Roque, Naples, FL (US); Justin Boyle, Naples, FL (US); Zachary Ingwer, Naples, FL (US); Timothy McIntyre, Naples, FL (US)

(21) Appl. No.: 19/415,246

(22) Filed: Dec. 10, 2025

Related U.S. Application Data
(60) Provisional application No. 63/730,581, filed on Dec. 11, 2024.

(51) Int. Cl. A61B 17/3205; A61B 17/00; A61B 17/32

(52) U.S. Cl. CPC A61B 17/3205; A61B 2017/00526; A61B 2017/320052

(57) ABSTRACT

A cutting instrument comprises a main body, an attachable blade set, and an adjustable cutting depth. A kit for the cutting instrument can include a plurality of blade sets for various cutting widths. A blade set selected for a desired cutting width is engageable with the main body.

### PRIOR APPLICATION

This application claims the benefit of U.S. Provisional Application No. 63/730,581 filed Dec. 11, 2024, the disclosure of which is incorporated by reference herein.

### BACKGROUND

This disclosure relates generally to surgical devices and methods and, more particularly, to devices and associated methods for extracting tissue for use in grafts.

Tendons are commonly harvested for use in orthopedic procedures. For example, tendons may be autografts harvested from a patient's hamstring tendon, quadriceps tendon, or other areas of the body. The harvested tendons may be used in ligament reconstruction surgeries, e.g., anterior cruciate ligament (ACL), posterior cruciate ligament (PCL), and ulna collateral ligament (UCL) reconstruction surgeries.

A challenge of a specialized device for tendon harvesting is ensuring that the harvested tissue is of the appropriate size for the implantation site. Because each individual patient has a unique physiology, manufacturers of such harvesting tools produce cutters of various sizes corresponding to the desired depth and width of the graft. Accordingly, suppliers and surgery centers need to maintain and manage inventory of the various-sized tools. In addition, surgical suites need to provision multiple sizes from which the surgeon will select an appropriate size for use during each the procedure. Managing the inventory of such tools adds to the complexity and cost of operating surgical practices and associated facilities.

### BRIEF SUMMARY

This disclosure relates to a surgical cutting instrument that is modular in its construction. The cutting instrument is configurable for various cutting depths and widths using snap-fit assembly of varying-size components. In some embodiments, the cutting instrument includes a main body, a blade set, and a depth stop. A main body may include a handle portion at a proximal end and an elongate portion extending from a base at the handle portion to a distal end. The main body can include mounting points for receiving and retaining the blade set and the depth stop.

In some embodiments, a handle portion may have a suitable form and size to facilitate gripping of the cutting instrument by a surgeon. In a related embodiment, portions of the main body are integrally formed as a unitary component, such as by injection molding of a thermoplastic material.

In an embodiment, a distal end of the elongate portion of the main body includes mounting points comprising a longitudinal channel having a slot opening facing the distal end. In another embodiment, the distal end further includes a boss portion protruding into the channel. The mounting points at the distal end may be operative to engage with, and retain, the blade set.

In an embodiment, the main body further includes a mounting point along the elongate portion operative to engage with, and retain, the depth stop.

A blade set can include a plurality of blades arranged in a spaced relationship that are attachable as a set to the main body. The spacing between the blades corresponds to the width of the grafts to be harvested. In some embodiments, the blade set has one pair of blades. The blade set may further include an attachment portion that is formed to engage with one or more mounting points of the main body. In one particular example, the attachment portion includes an extension. The extension may be sized to partially fit in the channel at the distal end of the elongate portion of the main body when inserted through the slot opening. In a related embodiment, the extension includes an aperture that is sized to engage with the boss portion protruding into the channel of the elongate portion of the main body.

In an embodiment, the blade set is formed from a sheet of suitable metal alloy, such as stainless steel, which may be stamped, bent, and machined to produce its overall shape and create the cutting edges of the blades.

In another embodiment, the extension of the blade set has bend that elastically deflects when the blade set is properly engaged with the main body to apply a biasing force that opposes disengagement.

The depth stop can provide a spacer that is situated between the blades of the blade set. In an embodiment, the depth stop includes a generally elongate body having a proximal end and a distal end. The proximal end includes a retention portion that engages with the mounting points along the elongate portion of the main body. In an embodiment, the distal end of the depth stop includes a retention portion that engages with blade set. When the depth stop is engaged with the main body and the blade set, the spacer provided by the depth stop effectively shortens the cutting depth of the blades according to the thickness of the depth stop's spacer.

In an embodiment, the retention portion at the proximal end includes a latch structure, such as a barb or a hook, which engages with the main body. The retention portion of the distal end of the depth stop may include a barb or hook that fits snugly over a protruding portion of the attachment portion of the blade set.

In an embodiment, a specific configuration of the cutting instrument may be assembled to meet the needs of a given surgical procedure. According to a method of assembly, a main body is provided. A blade set is selected having a specific width, i.e., blade spacing, from among different blade sets of varying widths, and the selected blade set is attached to the main body. The attachment may be secured using a snap-fit mechanism that facilitates the retention of the blade set's attachment portion by the mounting points of the main body.

Next, a depth stop having the desired spacer thickness is selected. The selected depth stop is attached to the assembly using engagement of the barb or hook at the distal end of the depth stop with the protruding portion of the extension portion of the attachment portion of the blade set, and the retention portion of the depth stop at its proximal end is engaged with the mating surfaces of the elongate portion of the main body.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn, are not necessarily intended to convey any information regarding the actual shape of the particular elements and may have been solely selected for ease of recognition in the drawings. The headings and Abstract of the Disclosure provided herein are for convenience only and do not interpret the scope or meaning of the embodiments.

FIG. 1 is an orthographic-view diagram illustrating a modular cutting instrument according to an example embodiment.

FIGS. 2-5 are a top-view, sectional view, distal-end-view, and bottom-view diagrams illustrating a main body of the modular cutting instrument of FIG. 1.

FIGS. 6-8 are diagrams illustrating blade set 104 from the side, top, and front views, respectively.

FIGS. 9-11 are top-view, side-view, and proximal-end-view diagrams, respectively, illustrating depth stop 106 according to an embodiment.

FIG. 12 is a perspective-view diagram illustrating a distal portion of a cutting instrument, which has a knob-adjustable cutting depth according to one type of embodiment.

FIG. 13 is a diagram illustrating the blade-positioning mechanism in greater detail.

FIGS. 14-15 are partial cross-sectional-view diagrams illustrating a distal end of a cutting instrument with an adjustable depth according to another type of embodiment that features a user-operable slide to set the cutting depth.

### DETAILED DESCRIPTION

### Modular Cutting Instrument.

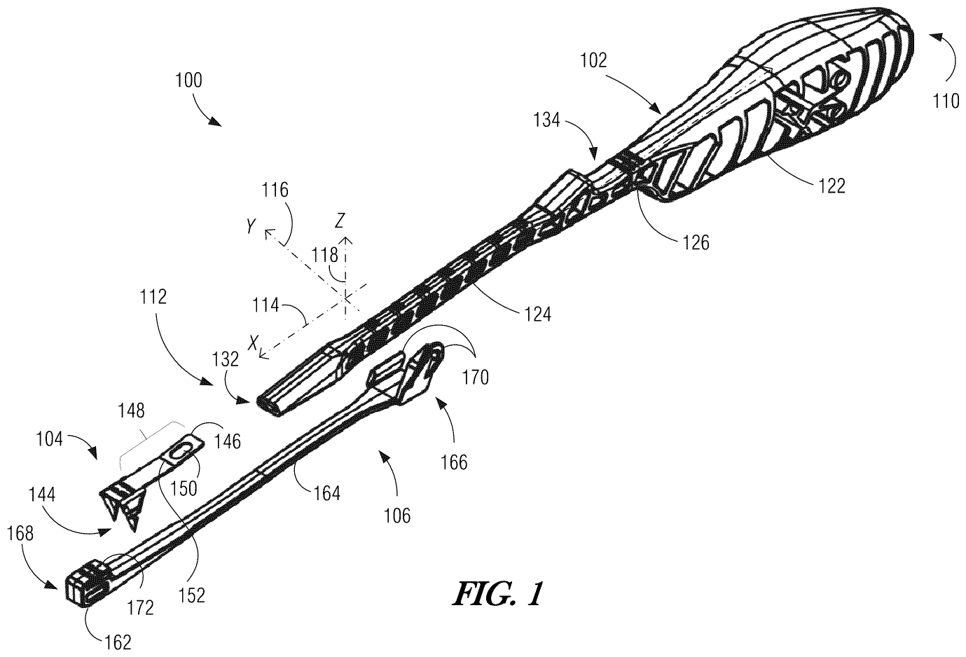

FIG. 1 is an orthographic-view diagram illustrating a modular cutting instrument according to an example embodiment. As detailed in the following description, cutting instrument 100, as depicted, is configurable for various cutting depths and widths using snap-fit assembly of varying-size components. Cutting instrument 100 includes main body 102, blade set 104, and depth stop 106, which are readily assembled together, without tools, to form a robust, reliable, and ready-to-use cutting instrument 100. Notably, blade set 104 may be available in various widths. Likewise, depth stop 106 may be available in various depths. Accordingly, for each specific patient procedure, cutting instrument 100 may be configured to provide a specific width and depth of cuts.

In the following description, cutting instrument 100 and its components are described with reference to proximal end 110, distal end 112, and reference coordinate system comprising x, y, and z axes 114, 116, and 118, respectively. X-axis 114 represents the longitudinal axis, along which proximal and distal ends 110 and 112, respectively, are defined. Y-axis 116 represents the transverse, or lateral, directions; and z-axis 118 represent the vertical axis in the present reference frame.

Main body 102 may be formed from a suitable material having desired hardness/rigidity, biocompatibility, and other characteristics. It may be formed from a thermoplastic, for instance, via an injection-molding process. In other examples, main body 102 may be co-molded from multiple different materials. Related embodiments may utilize an over-molding process which forms one or more portions of main body 102 over at least a portion of a substrate, frame, or skeleton structure. In some embodiments, a machining process may be performed to form certain features of main body 102. Various structural features may be included in main body 102 to enhance rigidity or facilitate flexibility, facilitate assembly and retention of the components of cutting instrument 100, and enhance ergonomics of cutting instrument 100.

In a related embodiment, main body 102 has handle portion 122 at proximal end 110. Handle portion 122 may be integrally formed with main body 102, as depicted, or it may be separately formed as an additional component (not shown) to be assembled with the other parts of instrument 100. Main body 102 also includes elongate portion 124 extending from base 126 at the distal end of handle portion 122. Main body 102 includes certain structural features, such as mounting points 132, 134 for receiving and retaining blade set 104 and depth stop 106, respectively.

In some embodiments, handle portion 122 may have a suitable size and shape to facilitate gripping of the cutting instrument by a surgeon. Handle portion 122 may include certain other features to support usability of instrument 100, such as having surface texture or a set of recesses or protrusions that reduce slipping of the instrument.

Mounting points 132 at distal end 112 may be operative to engage with, and retain, blade set 104. In one type of embodiment, mounting points 132 include a longitudinal channel having a slot with an opening that faces distally. In a related embodiment, mounting points 132 further include a catch protruding vertically. These features are described in greater detail below.

In an embodiment, main body 102 further includes mounting points 134 along the elongate portion that are operative to engage with, and retain, depth stop 106. In one implementation, mounting points 134 along the elongate portion include a planar top surface having defined corners to which proximal retention portion 170 of depth stop 106 can latch on.

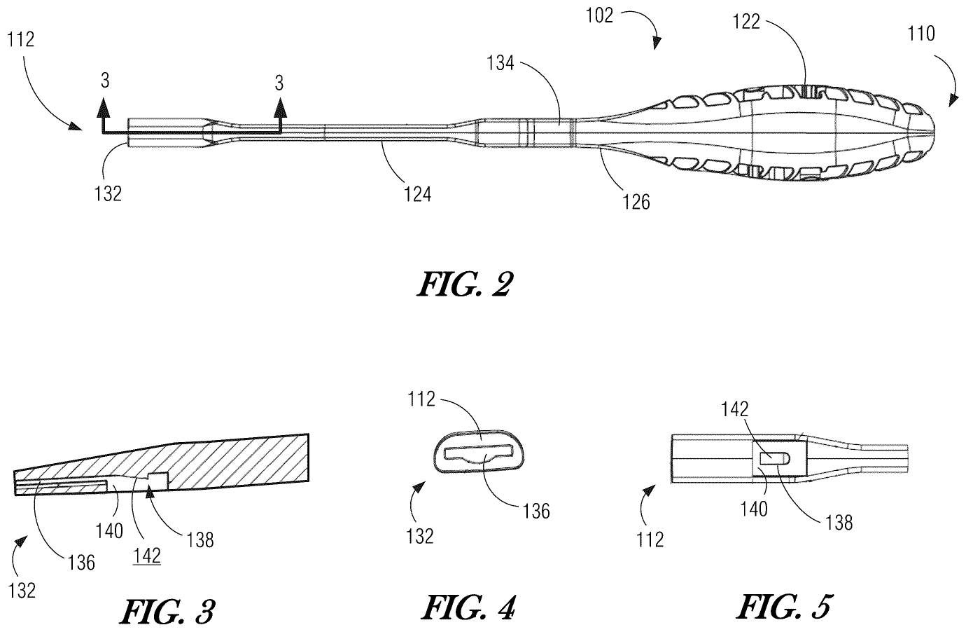

FIG. 2 is a top-view diagram illustrating main body 102, with several of the features described above indicated. Section 3-3, taken longitudinally at distal end 112, is shown in FIG. 3. As illustrated, mounting points 132 at distal end 112 include channel 136 and catch 138. FIG. 4 is a front-view diagram of distal end 112 showing a generally slot-shaped opening to channel 136. Channel 136 extends along the longitudinal axis 114 to channel exit 140, which is an opening in the bottom surface of distal end 112 of main body 102. FIG. 5 is a bottom-view diagram illustrating distal end 112, including and channel exit 140, and showing catch 138 in grater detail. Catch 138 has the shape of a vertical protrusion with an inclined surface 142. As explained below, channel 136 and catch 138 engage with extension 148 and aperture 150, respectively, to securely retain blade set 104.

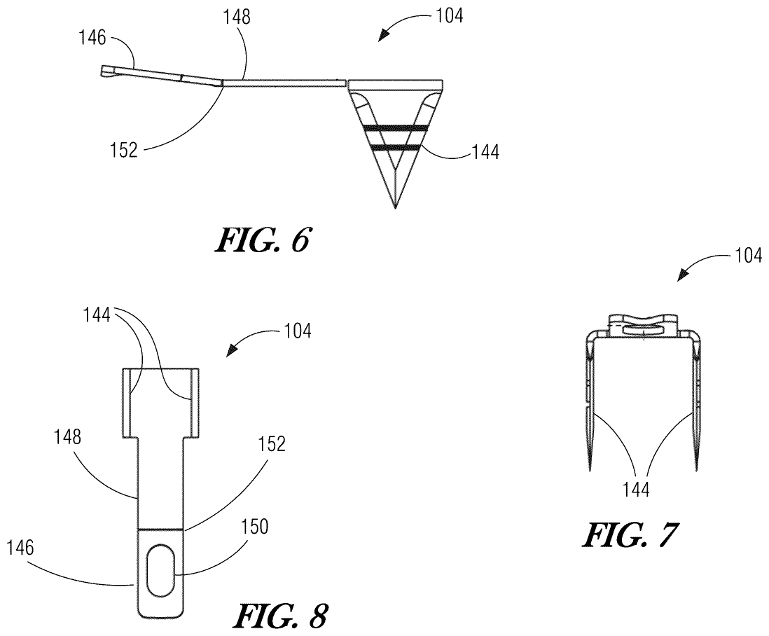

Blade set 104 includes a plurality of blades 144 to be attached to main body 102, having the blades arranged in a spaced relationship corresponding to a desired cutting width. FIGS. 6-8 are diagrams illustrating blade set 104 from the side, top, and front views, respectively. As depicted in this example, the plurality of blades 144 is a single pair of blades. The spacing between the blades along the transverse direction (y axis 116) corresponds to the width of the grafts to be harvested. In some embodiments, blade set 104 may further include attachment portion 146 that is formed to engage with mounting points 132 at distal end 112 of main body 102. In an embodiment, attachment portion 146 includes strip-shaped extension 148 along the longitudinal direction (x axis 114). Extension 148 may be sized to partially fit in channel 136 at distal end 112 of elongate portion 124 of main body 102 when inserted into the slot opening. In an embodiment, extension 148 includes aperture 150 that is sized to engage with catch 138 protruding into the channel of elongate portion 124 of main body 102.

In an blade set 104 is formed from a sheet of suitable metal alloy, such as stainless steel, which may be stamped, bent, or machined to produce its overall shape and create the sharp cutting edges of the plurality of blades 144.

In another related example, extension 148 of blade set 104 has bend 152 that elastically deflects when the blade set is properly engaged with catch 138 to apply a biasing force that latches aperture 150 to catch 138 and opposes disengagement.

Depth stop 106 provides spacer 162 that, when assembled as part of instrument 100, is situated proximate the blades 144 of blade set 104 and this position of spacer 162 limits the effective cutting depth of plurality of blades 144.

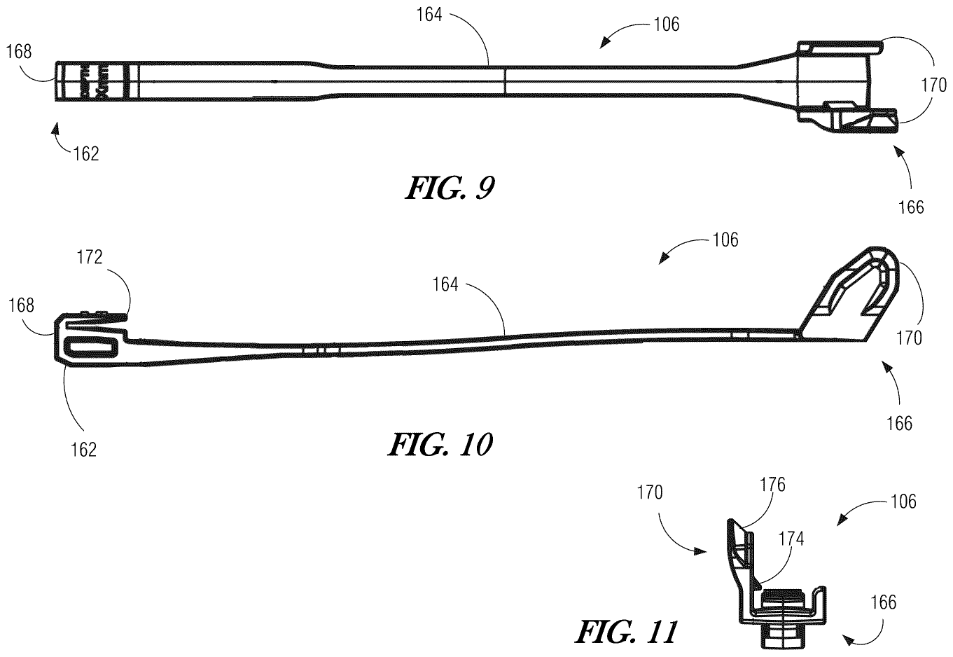

FIGS. 9-11 are top-view, side-view, and proximal-end-view diagrams, respectively, illustrating depth stop 106 according to an embodiment. In one type of embodiment, depth stop 106 includes a generally elongate body 164 having proximal end 166 and distal end 168, and is sized and shaped to conform to the bottom surface of main body 102. Proximal end 166 includes proximal retention portion 170 that engages with mounting points 134 along elongate portion 124 of main body 102. Proximal retention portion 170 is shown with greater detail in FIG. 11. In the implementation that is depicted, proximal retention portion 170 includes a latch 174 on a release lever 176. Release lever 176 is elastically deformable in the outward lateral direction to release latch 174.

In an embodiment, the distal end 162 of the depth stop includes distal retention portion 172 that engages with blade set 104. When depth stop 106 is engaged with main body 102 and blade set 104, spacer 162 provided by depth stop 106 effectively shortens the cutting depth of blades 144 according to the thickness along the vertical direction (i.e., z axis 118) of the depth stop's spacer 162.

Proximal retention portion 170 is shown with greater detail in FIG. 11. In the implementation that is depicted, proximal retention portion 170 includes a latch 174 on a release lever 176. Latch 174 engages with mounting points 134 of main body 102. Release lever 176 can be deflected laterally to release latch 174. Distal retention portion 172 is shown in greater detail in FIG. 10. Distal retention portion 172 at distal end 168 of depth stop 106 may include a clasp that fits snugly over a distal end of blade set 104.

Depth stop 106 may be formed as a single unit from a suitable thermoplastic using injection molding or other suitable fabrication technique, which may be automated and implemented at scale. Accordingly, the unit cost of depth stop 106, in terms of materials and labor is expected to be quite low.

According to a related aspect, a specific configuration of cutting instrument 100 may be assembled to meet the needs of a given surgical procedure. The specific configuration has a selected cutting width from a plurality of available cutting widths, and a selected cutting depth from a plurality of cutting depths. To provide the various cutting width and cutting depth options, a kit for cutting instrument 100 may be provided that includes main body 102, a diverse set of blade sets 104 having various blade spacings (i.e., cutting widths), and a diverse set of depth stops 106 having various heights of spacer 162 and various widths of spacer 162 to match the various blade spacings.

In one example, a kit includes several sets of blades, such as blade sets having cutting widths of 8 mm, 9 mm, 10 mm, and 11 mm. The kit may further include a variety of sizes of depth stop 106 corresponding to various spacer heights and blade widths of the blade sets included in the kit. Table 1 below indicates depth stop sizes for the various blade sets 104 and spacer heights of spacer 162 (in this example, a total of 8 depth stop sizes are provided as part of the kit):

TABLE 1
Depth Stops as part of example kit

Spacer Height

| | | |
|---|---|---|
| Blade Width | 6 mm | 8 mm |

| | | | |
|---|---|---|---|
| 8 | mm | X | X |
| 9 | mm | X | X |
| 10 | mm | X | X |
| 11 | mm | X | X |

More generally, in a kit with b different blade-set widths and h spacer heights, the number of depth stops of various sizes to be provided in order to support all combinations of cutting with and depth is b×h.

According to a method of assembly, main body 102 is provided. A specific blade set 204 is selected having a desired width, i.e., blade spacing, from among different blade sets of varying widths, and the selected blade set is attached to distal end 112 of main body 102. The attachment may be secured using a snap-fit mechanism that facilitates the retention of the blade set's attachment portion by the mounting points of the main body. In one such embodiment, attachment portion 146 of the selected blade set 104 is inserted into channel 136 from distal end 112. Most of the length of extension 148 (e.g., substantially all of extension 148) may be slid through channel 136 until aperture 150 engages with catch 138. Bend 152 causes attachment portion 146 to be biased in the upward direction such that aperture 150 remains securely engaged with catch 138.

If necessary, blade set 104 can be removed from main body 102 by deflecting attachment portion 146 in the downward direction (against the direction of bend 152) to disengage aperture 150 from catch 138, and then sliding blade set 104 outward in the distal direction through channel 136 until the parts are separated. Importantly, because attachment portion 146 is securely biased to keep aperture 150 engaged with catch 138, blade set 104 will not separate from main body 102 during a cutting operation in any direction.

Next, for assembly of cutting instrument 100, depth stop 106 having the desired thickness of spacer 162, and width of spacer 162 corresponding to the selected blade spacing, is selected. The selected depth stop 106 is attached to the assembly having blade set 104 secured to main body 102 using engagement of distal retention portion 172 at the distal end of the depth stop with the distally-protruding portion of blade set 104, and proximal retention portion 170 of depth stop 106 at its proximal end 166 is engaged with the mating surfaces 134 of elongate portion 124 of main body 102.

Advantageously, this assembly procedure achieves a cutting instrument 100 with a set cutting width and a set cutting depth, each of which was independently selected in the surgical suite. Moreover, because the structures described above facilitate assembly and disassembly of cutting instrument 100 easily and without the need for tools, the surgeon may configure and reconfigure cutting instrument 100 as needed at the time of the tendon-harvesting procedure.

Furthermore, thanks to the simplicity and low unit cost of each blade set 104 and depth stop 106, it may be economically feasible to provide a kit of various cutting widths of blade set 104 and various widths and depths of depth stop 106, together with main body 102 as a commercial unit of cutting instrument 100. Accordingly, distribution and inventory management of cutting instrument 100 is streamlined.

### Knob-Adjustable Cutting Depth.

FIG. 12 is a perspective-view diagram illustrating a distal portion of a cutting instrument, which has a knob-adjustable cutting depth according to one type of embodiment. As depicted, cutting instrument 200 includes main body 202, which holds blade set 204. Blade set 204 is generally similar to blade set 104 as described above, with a pair of spaced-apart cutting blades and an extension portion. The attachment end of the extension portion is affixed to main body 202 (affixation not specifically shown). The affixation may be implemented using any suitable technique, such as welding, adhesive, fastener, over-molding, or the like.

Cutting Instrument 200 also includes knob 206 at the top surface of main body 202 and threaded shaft 208 extending from knob 206 through the top surface of main body 202 and impinging on the extension portion of blade set 204. The threads of threaded shaft 208 are engaged with a threaded bore of main body 202 or a nut that is affixed to main body 202. Rotation of the knob advances or retreats the end of threaded shaft 208 in the vertical direction (i.e., perpendicular to the top surface of main body 202 through which threaded shaft 208 passes), which also elastically deflects the extension portion of blade set 204 to reposition the blades generally along the vertical direction. This repositioning effectively sets the cutting depth.

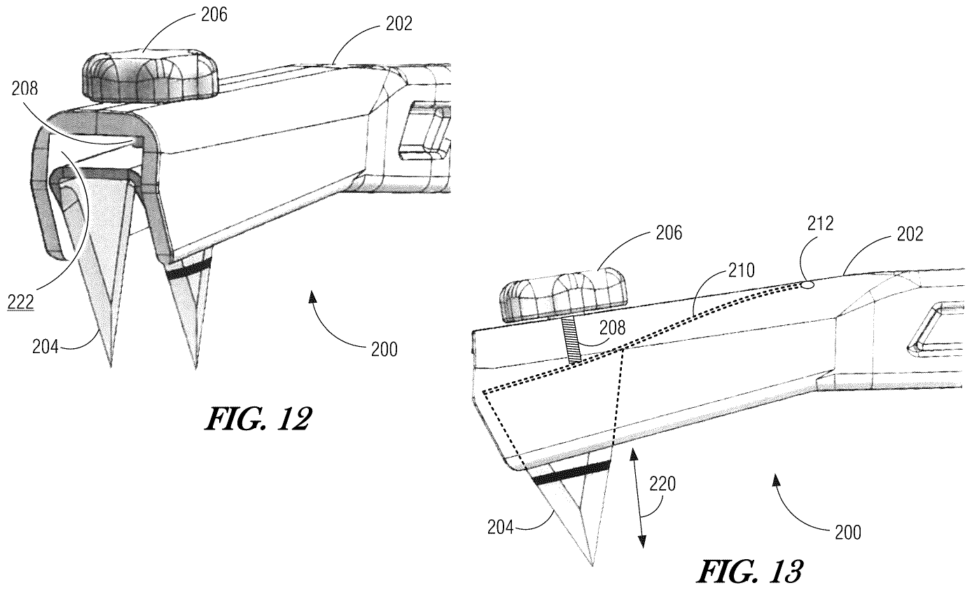

FIG. 13 is a diagram illustrating the blade-positioning mechanism in greater detail. As shown, extension portion 210 of blade set 204 attaches proximal end of extension portion 210 to main body 202 at attachment point 212. Attachment 212 may be achieved by overmolding extension portion 210 with main body 202, affixing extension portion 210 with a snap-fit arrangement (e.g., hook, barb, or pin that engages with a suitable mating feature such as an aperture, loop, recess, barb, or pawl)—not shown. Adjustment of knob 206 turns threaded shaft 208 and adjusts its position against extension portion 210. Cutting depth 220, which corresponds to the extent to which the cutting blades protrude from the bottom surface of main body 202, is thus adjustable.

In an embodiment, knob 206 is removable and re-attachable from/to threaded shaft 208. Removal of knob 206 after setting the cutting depth 220 provides a lower overall profile for instrument 200. In some embodiments, grooves or detents (not shown) may be formed in interior surface 222, with which a distal end of extension portion 210 engages. Such grooves or detents function to retain blade set 204 at its set cutting depth. The grooves or detents may be shaped as ratcheting, or asymmetrical teeth that, when engaged with extension portion 210, oppose retraction of blade set 204.

### Slide-Adjustable Cutting Depth.

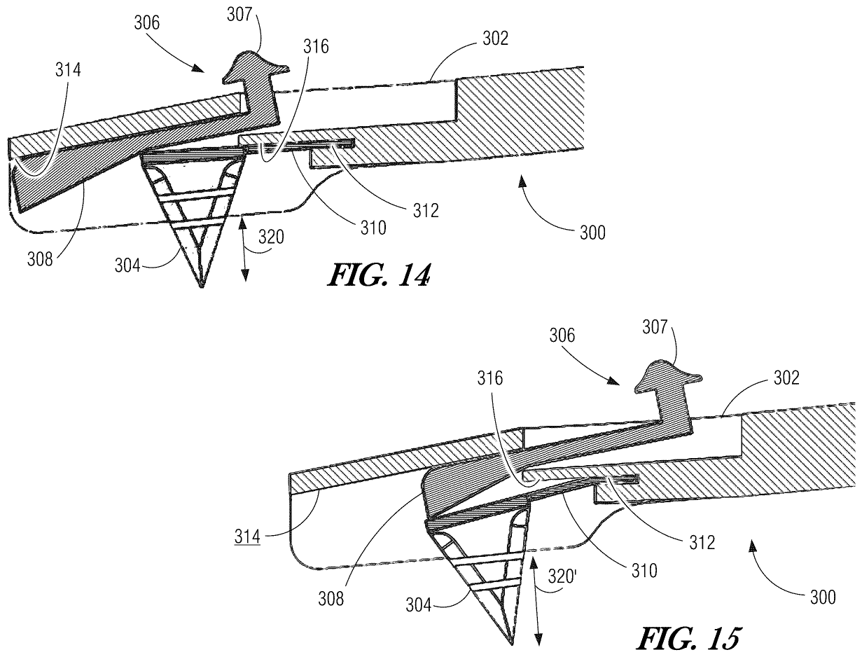

FIGS. 14-15 are partial cross-sectional-view diagrams illustrating a distal end of a cutting instrument with an adjustable depth according to another type of embodiment that features a user-operable slide to set the cutting depth. As depicted, cutting instrument 300 comprises main body 302, blade set 304 with extension portion 310 attached to main body 302 at attachment point 312 and arranged as a cantilever, and slide 306.

As depicted, slide 306 includes grip 307 protruding upwards above the exterior periphery of main body 302, and a wedge portion 308 extending distally (to the left as shown) from grip 307. Wedge portion 308 has a thicker height at the distal end. Wedge portion is generally situated between blade set 304 and interior slide surface 314 of main body 302.

As shown in FIG. 14, when slide 306 is in its forward (distal) position, a thinner part of wedge 308 is between blade set 304 and interior slide surface 314, which allows extension portion 310 to be in its nominal, non-deflected state against stop surface 316. When extension portion 310 is in this state, blade set 304 is in its fully-retracted position, such that the cutting depth 320 is minimal.

As shown in FIG. 15, when slide 306 is in its rearward (proximal) position, a thicker part of wedge 308 is between blade set 304 and interior slide surface 314, which causes extension portion 310 to elastically deflect downward away from stop surface 316. When extension portion 310 is in this state, blade set 304 is in its fully-extended position, such that the cutting depth 320′ is maximal. Extension portion 310, when flexed, is biased upward against wedge 308, thereby maintaining the set cutting depth.

Positioning slide 306 anywhere between the forward and rearward-most positions causes the cutting depth of blade set 304 to be commensurately set. In some embodiments, interior slide surface 314, wedge 308, or both, include detents or surface texture that tends to increase friction for holding the position of slider 306 in its desired position.

### ADDITIONAL NOTES AND EXAMPLES

Example 1 is a modular cutting instrument kit, comprising: a main body, an attachable blade set, and an attachable depth stop; the main body having a proximal end and a distal end, and further including: a handle portion at the proximal end; first mounting points, at the distal end, engageable with the blade set; and second mounting points engageable with the depth stop; the blade set comprising a plurality of blades and an attachment portion engageable with the first mounting points; and the depth stop comprising a spacer and at least one retention portion engageable with the second mounting points, the spacer having a first height; wherein the blade set and the depth stop, when attached to the main body via the first and the second mounting points, form a cutting instrument in which a cutting depth of the blade set is limited based on the first height of the spacer.

In Example 2, the subject matter of Example 1 includes, wherein the attachable blade set is removably attachable to the main body at the first mounting points and the attachable depth stop is removably attachable to the main body at the second mounting points.

In Example 3, the subject matter of Examples 1-2 includes, wherein depth stop is attachable to the blade set when the blade set is attached to the main body in addition to being attachable to the second mounting points.

In Example 4, the subject matter of Examples 1-3 includes, wherein the at least one retention portion includes a latch adapted to engage with the second mounting points.

In Example 5, the subject matter of Example 4 includes, wherein the at least one retention portion further includes a release lever elastically deformable operable to release the latch.

In Example 6, the subject matter of Examples 1-5 includes, wherein the second mounting points are situated proximate the handle portion.

In Example 7, the subject matter of Examples 1-6 includes, wherein the blade set and the depth stop are each attachable to the main body without tools.

In Example 8, the subject matter of Examples 1-7 includes, wherein the elongate portion of the main body is integrally formed with the handle portion.

In Example 9, the subject matter of Examples 1-8 includes, wherein the blade set comprises a pair of parallel blades spaced apart at a defined first width.

In Example 10, the subject matter of Examples 1-9 includes, a plurality of additional blade sets having respective pairs of parallel blades spaced apart at different widths.

In Example 11, the subject matter of Examples 1-10 includes, a plurality of additional depth stops having respective spacers of various widths and heights.

In Example 12, the subject matter of Examples 1-11 includes, wherein the first mounting points include a channel defined in the main body with an opening sized to receive and retain the attachment portion of the blade set.

In Example 13, the subject matter of Example 12 includes, wherein the first mounting points further include a catch protrusion positioned to engage with the attachment portion of the blade set.

In Example 14, the subject matter of Example 13 includes, wherein the attachment portion of the blade set includes a strip-shaped extension sized to slidably fit into the channel.

In Example 15, the subject matter of Example 14 includes, wherein the extension defines an aperture that is sized and positioned to engage with the catch protrusion when the extension is fully inserted into the channel.

In Example 16, the subject matter of Example 15 includes, wherein the extension includes a bend arranged to effect an elastic deflection of the extension when the extension is fully inserted into the channel, the elastic deflection causing a biasing force of the extension that securely latches the aperture to the catch protrusion.

Example 17 is a method for assembling a modular cutting instrument, the method comprising: providing a main body comprising a proximal end having a handle and a distal end, a plurality of blade sets having respective pairs of cutting blades at various spacings, and a plurality of depth stops having respective spacers of various widths and heights; selecting a first blade set from the plurality of blade sets, the first blade set comprising a first pair of cutting blades at a desired spacing; selecting a first depth stop from the plurality of depth stops, the first depth stop comprising a first spacer having a width corresponding to the desired spacing of the cutting blades of the selected first blade set, and a height corresponding to a desired cutting depth; removably attaching the first blade set to the distal end of the main body; and removably attaching the first depth stop to the main body, wherein the first spacer is situated between the first pair of cutting blades.

In Example 18, the subject matter of Example 17 includes, removably attaching the first depth stop to the first blade set.

In Example 19, the subject matter of Examples 17-18 includes, wherein removably attaching the first blade set to the distal end of the main body includes latching the first blade set to the main body without use of any additional tools.

In Example 20, the subject matter of Examples 17-19, includes, wherein removably attaching the first depth stop to the main body includes conforming the first depth stop to a surface of the main body and latching the first depth stop to the main body without the use of any additional tools.

Example 22 is an apparatus comprising means to implement of any of Examples 18-20.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The various embodiments described above can be combined to provide further embodiments.

The methods described herein can be performed with variations. For example, many of the methods may include additional acts, omit some acts, and/or perform acts in a different order than as illustrated or described.

As used herein, “greater than” and “less than” limits may also include the number associated therewith. Stated another way, “greater than” and “less than” may be interpreted as “greater than or equal to” and “less than or equal to.” It is contemplated that this language may be subsequently modified in the claims to include “or equal to.” For example, “greater than 50 percent” may be interpreted as, and subsequently modified in the claims as “greater than or equal to 50 percent.”

In some embodiments, any or some of the components or steps disclosed herein may be considered optional. In some cases, the disclosed assemblies may expressly exclude any or some of the aforementioned elements or steps in this description, e.g., via claim language. Such negative limitations are contemplated, and this text serves as support for negative limitations for components, steps, and/or features.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A cutting instrument kit, comprising:
a main body, an attachable blade set, and an attachable depth stop;
the main body having a proximal end and a distal end, and further including:
a handle portion at the proximal end;
first mounting points, at the distal end, engageable with the blade set; and
second mounting points engageable with the depth stop;
the blade set comprising a plurality of blades and an attachment portion engageable with the first mounting points; and
the depth stop comprising a spacer and at least one retention portion engageable with the second mounting points, the spacer having a first height;
wherein the blade set and the depth stop, when attached to the main body via the first and the second mounting points, form a cutting instrument in which a cutting depth of the blade set is limited based on the first height of the spacer.

2. The cutting instrument kit of claim 1, wherein the attachable blade set is removably attachable to the main body at the first mounting points and the attachable depth stop is removably attachable to the main body at the second mounting points.

3. The cutting instrument kit of claim 1, wherein the depth stop is attachable to the blade set when the blade set is attached to the main body in addition to being attachable to the second mounting points.

4. The cutting instrument kit of claim 1, wherein the at least one retention portion includes a latch adapted to engage with the second mounting points.

5. The cutting instrument kit of claim 4, wherein the at least one retention portion further includes a release lever elastically deformable operable to release the latch.

6. The cutting instrument kit of claim 1, wherein the second mounting points are situated proximate the handle portion.

7. The cutting instrument kit of claim 1, wherein the blade set and the depth stop are each attachable to the main body without tools.

8. The cutting instrument kit of claim 1, wherein the elongate portion of the main body is integrally formed with the handle portion.

9. The cutting instrument kit of claim 1, wherein the blade set comprises a pair of parallel blades spaced apart at a defined first width.

10. The cutting instrument kit of claim 1, further comprising a plurality of additional blade sets having respective pairs of parallel blades spaced apart at different widths.

11. The cutting instrument kit of claim 1, further comprising a plurality of additional depth stops having respective spacers of various widths and heights.

12. The cutting instrument kit of claim 1, wherein the first mounting points include a channel defined in the main body with an opening sized to receive and retain the attachment portion of the blade set.

13. The cutting instrument kit of claim 12, wherein the first mounting points further include a catch protrusion positioned to engage with the attachment portion of the blade set.

14. The cutting instrument kit of claim 13, wherein the attachment portion of the blade set includes a strip-shaped extension sized to slidably fit into the channel.

15. The cutting instrument kit of claim 14, wherein the extension defines an aperture that is sized and positioned to engage with the catch protrusion when the extension is fully inserted into the channel.

16. The cutting instrument kit of claim 15, wherein the extension includes a bend arranged to effect an elastic deflection of the extension when the extension is fully inserted into the channel, the elastic deflection causing a biasing force of the extension that securely latches the aperture to the catch protrusion.

17. A method for assembling a cutting instrument, the method comprising:
providing a main body comprising a proximal end having a handle and a distal end, a plurality of blade sets having respective pairs of cutting blades at various spacings, and a plurality of depth stops having respective spacers of various widths and heights;
selecting a first blade set from the plurality of blade sets, the first blade set comprising a first pair of cutting blades at a desired spacing;
selecting a first depth stop from the plurality of depth stops, the first depth stop comprising a first spacer having a width corresponding to the desired spacing of the cutting blades of the selected first blade set, and a height corresponding to a desired cutting depth;
removably attaching the first blade set to the distal end of the main body; and
removably attaching the first depth stop to the main body, wherein the first spacer is situated between the first pair of cutting blades.

18. The method of claim 17, further comprising:
removably attaching the first depth stop to the first blade set.

19. The method of claim 17, wherein removably attaching the first blade set to the distal end of the main body includes latching the first blade set to the main body without use of any additional tools.

20. The method of claim 17, wherein removably attaching the first depth stop to the main body includes conforming the first depth stop to a surface of the main body and latching the first depth stop to the main body without the use of any additional tools.
